(19) United States
(12) Patent Application Publication
(10) Pub. No.: US 2011/0294228 A1
(43) Pub. Date: Dec. 1, 2011

(54) DETECTION DEVICES AND METHODS

(76) Inventors: Ronald R. Bowsher, Indianapolis, IN (US); Randy Aleks Davis, Indianapolis, IN (US)

(21) Appl. No.: 13/144,169

(22) PCT Filed: Jan. 14, 2010
(86) PCT No.: PCT/US10/21014

§ 371 Date: Aug. 23, 2011

Related U.S. Application Data
(60) Provisional application No. 61/145,169, filed on Jan. 16, 2009.

(51) Int. Cl. G01N 33/53; C12M 1/34

(52) U.S. Cl. 436/501; 422/69; 435/287.2

(57) ABSTRACT

The present invention relates to devices and methods for rapid and easy to use quantitative detection of one or more analytes in, for example, food, food ingredients, drinking water or pharmaceuticals. Analytes that can be detected by the devices and methods herein include, for example, adulterants, toxins, allergens, pathogens, pesticides, pharmaceuticals, pharmaceutical intermediates, biopolymers and biotechnology products.

This patent application claims priority to U.S. provisional application Ser. No. 61/145,169, filed Jan. 16, 2009, the entirety of which is hereby incorporated herein by reference.

### FIELD OF THE INVENTION

The present invention relates to devices and methods for rapid and easy to use quantitative detection of one or more analytes in, for example, food, drinking water or pharmaceuticals. Analytes that can be detected by the devices and methods herein include, for example, adulterants, toxins, allergens, pathogens, pesticides, pharmaceuticals, biopolymers and biotechnology products.

### BACKGROUND OF THE INVENTION

There is a significant public safety concern regarding food containing adulterants, toxins, allergens and other impurities.

For example, there is a significant public safety concern regarding the adulteration of human food and animal feed stock by addition of triazine moieties, including melamine and cyanuric acid. For example in 2008, melamine and cyanuric acid contaminated infant formula resulted in the death and hospitalization of numerous infants in Asia. In China, six babies likely died and nearly 300,000 have suffered urinary problems from drinking melamine-tainted milk powder. In the United States in 2008, the Food and Drug Administration (FDA) advised against human consumption of a number of food products including certain milk, chocolate, biscuit and coffee products because of possible melamine contamination. Furthermore, in 2008, the FDA reported finding trace amounts of melamine and cyanuric acid in infant formula marketed in the U.S.

In 2007, animal feed contaminated with melamine was discovered in fish and livestock feed in the U.S. Additionally in 2007, pet food adulterated with melamine led to the death of roughly 5,000 animals in the United States and many more in other countries outside the US, including Europe and Canada. Accordingly, there is an urgent need for devices and methods to allow rapid detection of triazine moieties, including melamine and cyanuric acid, in products for human or animal ingestion.

In addition to melamine and cyanuric acid, significant public safety risks are associated with many other adulterants, toxins and allergens contained in, for example, human food and animal feed.

Accordingly, there is an urgent need for devices and methods permitting an untrained user to rapidly and quantitatively detect harmful analytes that may be contained in a variety of different food and other products.

### SUMMARY

The present invention relates to devices and methods for rapid and easy to use quantitative detection of one or more analytes in, for example, food, food ingredients, drinking water or pharmaceuticals. The term “food” is intended to include human food, pet food and animal feed. The term pharmaceuticals is intended to include human, animal and mammalian medications, pharmaceutical drugs, intermediates and nutraceuticals.

Analytes that can be detected by the devices and methods herein include, for example, adulterants, toxins, allergens, pathogens, pesticides, pharmaceuticals, biopolymers and biotechnology products.

### BRIEF DESCRIPTION OF THE FIGURES

FIG. 1A is a front elevational view of a triazine detection device embodiment.

FIG. 1B is a front elevational view of a triazine detection device embodiment upon activation by a user.

FIG. 2A-D are schematics of direct competitive antibody reactions to detect analyte.

FIG. 3A-D are schematics of indirect competitive antibody reactions to detect analyte.

FIG. 4 is a horizontal cross-sectional view from above of a carrier material casing and solid support embodiment.

### DETAILED DESCRIPTION

The present invention provides simple to use, rapid and sensitive devices and methods for quantitative detection of one or more analytes in, for example, food, pharmaceuticals or drinking water. It is understood that the devices and methods herein are used to determine both the presence of analyte and also the absence of analyte below a predetermined amount. Accordingly, it is understood that the use of the term “detection” or “determining the presence of” herein is meant to include both of these aspects.

In certain embodiments, the devices are portable and in further embodiments, the devices are hand-held. In yet further embodiments, the devices are pocket-sized and/or weigh less than 0.5 kilograms, less than 1 kilogram or less than 2 kilograms.

Regarding analytes, the devices and methods can be used to detect, for example, adulterants, toxins, allergens, pathogens, pesticides, pharmaceuticals, pharmaceutical intermediates or ingredients, biopolymers and biotechnology products.

With regard to toxins, the devices and methods can be used to detect, for example, aflatoxin (mycotoxins), amatoxin, ergotamine, fumonisin, vomitoxin, rancidity (histamine), dioxins and toxins from the following pathogens: anthrax, clostridium, C. difficile (e.g., A & B), Salmonella, E. Coli and Pseudomonas.

With regard to allergens, the devices and methods can be used to detect, for example, penicillin, fungi, sulfonamides, and wheat gluten as well as other human, animal or mammalian allergens.

With regard to pathogens, the devices and methods can be used to detect, for example, anthrax, clostridium, C. difficile (e.g., A & B), Salmonella, E. Coli, Pseudomonas as well as other human, animal or mammalian pathogens.

With regard to adulterants, the devices and methods can be used to detect, for example, triazine moieties. The term “triazine moiety,” as used herein, refers to an unsubstituted or substituted heterocyclic 6 atom ring containing three carbon atoms and three nitrogen atoms and all salts, tautomers and hydrates thereof. Such compounds include, but are not limited to, halogen substituted, nitrogen substituted, or oxygen substituted triazine, or melamine, cyanuric acid, melamine cyanurate, ammeline, ammelide, benzoguanamine, cyanuric chloride, and all isomers, salts and hydrates thereof.

The term “triazine moiety” also includes all tautomers of the above compounds as exemplified below for cyanuric acid, melamine, ammeline and ammelide. An additional food adulterant than can be detected by the devices and methods described herein is glycosaminoglycan.

Referring to cyanuric acid, this molecule has been found to readily convert into two tautomeric forms shown in Formula I.

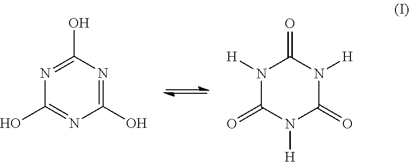

Referring to melamine, this molecule, in the solid state, has been found to predominantly exist in the form shown in Formula II.

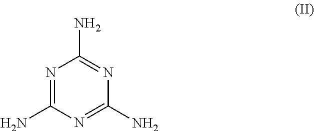

Melamine, in solution phase, has been found to exist in a tautomeric equilibrium as shown in Formula III.

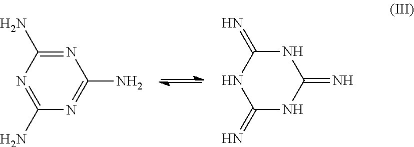

Ammeline and ammelide have been found to exist in tautomeric forms as shown in Formulas IV and V, respectively.

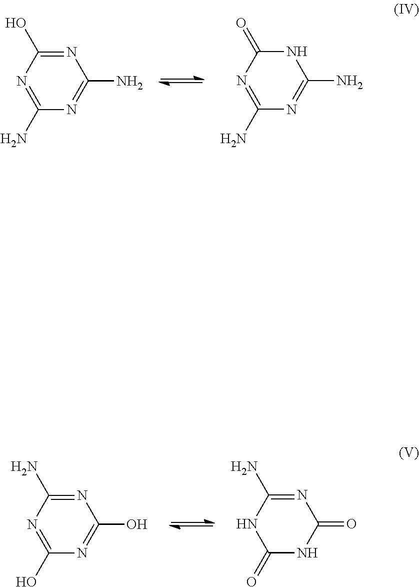

In certain embodiments, the devices and methods may be used to, for example, simultaneously or approximately at the same time, detect two or more, four or more or eight or more analytes in a single device, using, for example, two or more, four or more, or eight or more different carrier materials or the same number of different detection zones within one or more, two or more, three or more or four or more carrier materials. As an example, the device may contain four two-sided test strips thus providing for the detection of 8 different analytes.

The detection devices and methods, because of their design, use of non-toxic reagents, fast turnaround, and analytical performance characteristics (i.e., sensitivity and specificity) are appropriate for a use outside of a laboratory by a user who is not trained in the use of and does not have access to antibodies, laboratory chemicals or laboratory storage, mixing, washing, disposal devices or detection devices (such as photometers). Similarly, the detection devices and methods herein are suitable for a user who lacks technical training and experience and does not have access to routine laboratory equipment or capability and protective equipment for using caustic, toxic, hazardous or carcinogenic chemicals.

In an embodiment, to accommodate a plurality of matrices, the device incorporates a sample receiving port that is designed to accurately measure and hold a sample present in any form such as solid, liquid or semi-solid, and a sample mixing area that allows a sample present in any form to be readied for testing without any pre-treatment.

In an embodiment, the sample receiving port accurately measures and holds a predetermined amount of sample, without, for example, the use of a specialized weighing instrument. In an embodiment, the sample receiving port is sized to hold, when filled, the pre-determined amount. In another embodiment, the sample receiving port is marked to indicate a predetermined fill level. The sample receiving port further includes a cover that receives sample in an open position and then is movable to a closed position which introduces the sample into the sample mixing area. Also, the closed position and the open position of the sample receiving port create a barrier that seals in the contents of the sample mixing area.

In a further embodiment, it is contemplated that the device may include or may be used with a stick shaped spoon or other measurement/piercing apparatus to assist in locating the sample into the sample mixing area.

In embodiments of the invention, the sample is drinking water, liquid milk, powdered milk, infant formula, juice, chocolate, coffee, powdered or liquid dietary supplements, animal feed or any other food product in solid, semi-solid or liquid form or in any other form.

The sample mixing area incorporated in the detection device of the present invention allows a sample present in any form such as solid, liquid or semi-solid, to be tested without any pre-treatment. Although, not requiring pre-treatment, a solid particulate sample may be ground or crushed into smaller pieces, to more easily fit inside the sample receiving port. The sample mixing area facilitates mixing and dilution of the sample upon addition of the sample from the sample receiving port into the sample mixing area. In an embodiment, the reagent solution mixed with the sample in the sample mixing area dissolves a non-liquid sample such as a solid or a semi-solid sample and extracts triazine moieties present in the sample into the solution phase. The sample mixing chamber may be modified, as needed, to maximize analyte dissolution of and create a diluted homogenous sample. For example, the physical dimensions, shape and design can be modified to facilitate sample dissolution upon mixing by inversion as a means to create a homogenous testing milieu. In addition, non-reactive components, such as beads, can be included inside the mixing chamber to facilitate mixing and dissolution.

Advantages of embodiments of the present invention over conventional Enzyme-linked immunosorbant assay (ELISAs), other immunoanalytical and immunodiagnostic methodologies and other conventional detection devices and methods include (1) no requirement for technical training or experience, (2) no specialized equipment or pipetting devices and no requirement for separate chemical sample or reagent pre-treatment of the test sample, (3) no requirement for one or more separate washing steps, (4) no requirement for separate chemical storage containers or chemical disposal units, (5) the lack of hazardous chemicals, and (6) the ability to obtain results rapidly (within 5, 10, 15, 30, 45 or 60 minutes from the introduction of the sample into the sample mixing area), in a single device, and outside of a laboratory setting or environment, with quantitative results indicating the presence or absence of analyte at concentration levels that are harmful for human or animal consumption.

In a further embodiment, the device incorporates a reagent solution and a carrier material, wherein the carrier material comprises triazine moieties and/or antibodies that specifically bind to a triazine moiety. In a further embodiment, the triazine moieties and the antibodies that specifically bind to a triazine moiety participate in a direct or an indirect competitive reaction in the presence of a triazine-containing sample. In a further embodiment, depending on the type of a competitive reaction employed in the detection device, either the triazine moieties or the antibodies are labeled with visible signal producing moieties in such a way that, when the carrier material is contacted with the reagent solution and a sample comprising triazine moieties, a visible signal, such as color, or visible physical change is produced, and the signal is readable by a user with the unaided eye and/or without the use of a photometer or other specialized equipment. Examples of a visible physical change include a change (such as a decrease) in visible agglutination or precipitation produced, for example, using techniques such as immunogold-silver staining or colloid gold. In a further embodiment, the devices can be used by a person outside of a laboratory, and who is untrained and inexperienced in conducting immunoassays or other common analytical techniques.

Examples of color producing moieties include, but are not limited to, enzyme-substrate combinations, chromogenic dyes and colloidal gold. Enzyme labels that can be used include horseradish peroxidase (HRP), alkaline phosphatase (ALP), and urease.

The detection devices of the current invention are further capable of decreasing background interference from predetermined compounds such as, but not limited to, atrazine, purines or pyrimidines, or derivatives thereof to limit false-positive results. For example, in an embodiment, antibodies incorporated in the detection device bind with specificity to predetermined triazine moieties, for example, melamine and/or cyanuric acid, but not to atrazine, purine or pyrimidine moieties.

In a further embodiment, the device is designed to increase stability and decrease contamination. As an example, the reagent solution comprises a plurality of substances such as, but not limited to, buffering agents for pH control, salts for maintaining ionic equilibrium, antioxidants and stabilizing agents to increase the stability of reagents and detergents and antimicrobial compounds to decrease contamination and organics and chaotropic agents to increase solubility and minimize matrix interference.

Various buffering agents such as HEPES (4-(2-hydroxyethyl)-1-piperazineethanesulfonic acid), TRIS (Tris(hydroxymethyl)aminomethane), TES (N-Tris(hydroxymethyl)methyl-2-aminoethanesulfonic acid), BIS-TRIS (Bis(2-hydroxyethyl)amino-tris(hydroxymethyl)methane) and sodium borate can be used in the reagent solution. Salts such as sodium chloride, calcium chloride and potassium chloride can be used to maintain ionic equilibrium of the reagent solution. Examples of detergents that can be incorporated in the reagent solution include Triton X-100, Tween 20, and Tween 80, Antimicrobial compounds such as EDTA (ethylene diamine tetraacetic acid), EGTA (ethylene glycol tetraacetic acid) or Kathon can be used to prevent microbial contamination. Antioxidants such as citric acid and ascorbic acid can be incorporated to prevent oxidation of components of the reagent solution.

Various stabilizing agents such as bovine serum albumin (BSA), casein, poly-lysine, glycerol, sucrose, mannose, various protease inhibitors, carrier immunoglobulins and normal serum obtained from species such as horse, rat, mouse, chicken and fetal bovine serum can be used to maintain stability of antibodies present in the device. Examples of organics to increase solubility include alcohols, such as methanol, ethanol and isopropyl, and dimethylsulfoxide and dimethylformamide. Chaotropic agents, such as urea, guanidinium chloride, magnesium chloride, and lithium perchlorate may be added to increase dissolution and reduce interference from the test sample's matrix components.

The carrier material incorporated in an embodiment of the present invention comprises a plurality of triazine moieties and/or a plurality of antibodies that specifically bind to a triazine moieties. In an embodiment, the carrier material, for example, a test strip, is made up of a sorbent or porous material that facilitates a defined flow of the reagent solution mixed with a sample. In an embodiment, carrier material can be made of nitrocellulose, nylon, polyethersulfone, polyethylene, fused silica or crosslinked polyacrylate salts, inert beads or other forms of solid phases.

In an embodiment of the invention, coupling of antibodies or triazine moieties to the carrier material is via covalent bonding. In an embodiment, the carrier material is chemically modified to incorporate a reactive group(s) such as an aldehyde group and is reacted with an amino group(s) present on an antibody or a triazine moiety to covalently couple the antibody or the triazine moiety to the carrier material. In another embodiment, coupling is non-covalent, for example, by adsorption. Adsorption involves non-covalent interactions such as polar or non-polar interactions, electrostatic interactions, hydrogen bonding interactions or hydrophobic interactions. In a further embodiment, coupling immobilizes an antibody and/or a triazine moiety onto the carrier material.

In a further embodiment, the carrier material and the antibodies and/or triazine moieties coupled thereto are in a dry or liquid form and are not in liquid contact with the reagent solution.

The antibodies encompassed in an embodiment of the present invention are engineered or selected to bind specifically to a specific triazine moiety or to a class of triazine moiety. Thus, the antibodies encompassed by the present invention are capable of specifically binding to compounds such as, but not limited to, melamine, cyanuric acid and melamine cyanurate.

In an embodiment, the antibodies can be monoclonal or polyclonal. Furthermore, the antibodies can be immunoglobulins of any class and subtype or fragments thereof, or synthetic binding proteins such as single chain antibodies or diabodies. Alternatively, non-protein binding moieties may be used, for example, synthetic nucleotides, such as, aptamers. An aptamer is a nucleotide sequence having a three-dimensional configuration that binds specifically to a molecular target of interest.

In an embodiment, device parameters are (1) 0.01 g to 1 g of solid or semi-solid sample or 0.01 ml to 1 ml of liquid sample, (2) 1 to 5 ml of reagent solution, (3) 3 to 10 cubic cm of mixing space in the sample mixing area and (4) antibody affinity (K_{D}) of less than 10⁻⁹ M or an antibody affinity and specificity to detect at least 1 to 10 parts per billion of analyte in diluted sample (in this embodiment, the antibody detection of analyte in the diluted sample is 100 to 1,000 times greater than a desired undiluted sample detection level of at least 1 part per million (ppm). In a further embodiment, the antibody detection of analyte in the diluted sample is 50 to 100 times greater than the desired undiluted sample detection level. For example, if it is desired to detect analyte at 1 ppm, then the detection level for analyte in the diluted sample can be 10 part per billion (ppb) to 20 ppb or more. In a further embodiment, the amount of desired undiluted sample detection level is from at least 1 ppm to at least 2.5 ppm.

As a further example, if it is desired to detect an amount of analyte present at a concentration of at least 1 part per million (ppm) in undiluted sample, the following parameters are provided (1) 0.1 g solid or semi-solid sample or 0.1 ml of liquid sample, (2) 2.5 ml of reagent solution and (3) antibody specificity to detect 10 parts analyte per billion in the diluted sample.

It is understood that the parameters for the device are determined by a number of factors. First, the fill size of the sample receiving port and the amount of reagent solution provide a predetermined dilution. In the above example, a dilution ratio of 1:25 (sample:diluted sample) is provided. However, it is noted that the optimal dilution ration will be determined on a case-by-case manner for particular analytes and samples. The parameters for the fill size of the port and for the amount of dilution takes into account the desire to have a small and easily held and manipulated device, but at the same time a sufficient amount of dilution to dilute interfering chemicals present in the sample. Second, the size of the sample mixing area is designed to permit a sufficient amount of free space to allow for mixing to thoroughly dissolve the analyte in the reagent solution. Third, the size of the device as a whole is designed so that it can be easily held, for example, the device as a whole, in an embodiment, has a height of less than 5 to 25 cm, a width less than 3 to 8 cm and a depth less than 0.5 to 3 cm.

In another embodiment of the invention, the contents of the device are stable in the device for an extended period of time, for example, up to 2 years, 1 year, or 6 months. In a further embodiment, the device further is designed to stand up when placed on a flat surface (e.g., a table) and the device can include a fixed or detachable base for this purpose.

In a further embodiment of the invention, a plurality of triazine moieties present in the reagent solution are labeled with color producing moieties. In the presence of a sample containing triazine moieties, triazine moieties present in the sample compete with labeled triazine moieties in the reagent solution for binding to antibodies present on the carrier material. In the presence of triazine moiety in the sample, such competition results in a reduction in the amount of labeled triazine moiety binding and a consequent decrease in the intensity of visible signal or visible physical change observed.

In a further embodiment of the invention, a plurality of triazine moieties are coupled to a carrier material while a plurality of antibodies that specifically bind to a triazine moiety are present in the reagent solution. In the presence of a sample containing triazine moieties, triazine moieties present in the sample compete with the triazine moieties coupled to the carrier material for binding to antibodies that specifically bind to triazine moiety (primary antibody). The presence of a triazine moiety in the sample is detected by addition of a labeled secondary antibody or reagent that binds to the primary antibody bound to a triazine moiety on the carrier material. In the presence of triazine moiety in the sample, such competition results in a reduction in the amount of labeled triazine moiety binding and a consequent decrease in the intensity of visible signal or visible physical change observed.

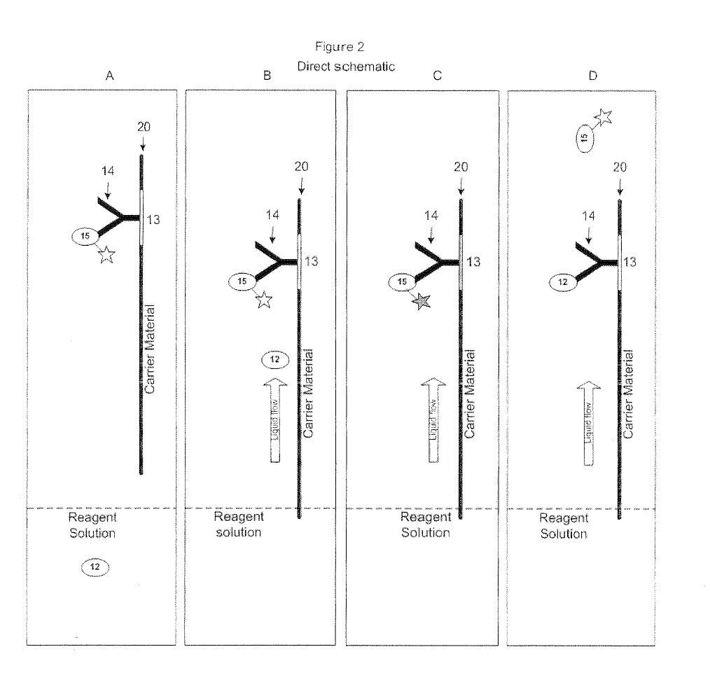

In a further embodiment of the invention, referring to FIG. 2, a carrier material 20 comprises an antibody 14 that specifically binds to a triazine moiety. The antibody 14 is coupled to the carrier material 20 at a detection region 13. In the embodiment, also coupled to the carrier material 20 at region 13 is a labeled triazine moiety 15 bound to antibody 14. Upon addition of a sample, the sample and the reagent solution will migrate through the carrier material 20 to the detection region 13 by, for example, capillary action. Triazine moiety 12 present in a sample and which is specifically bound by antibody 14 will compete with the labeled triazine moiety 15 on the carrier material for binding to antibody 14 (FIG. 2D). Such a competition will result in a decrease in the intensity of the signal observed in comparison with the signal that is observed in the absence of triazine moieties in the sample. In the absence of a triazine moiety 12 in a sample, there will not be a competition for binding to antibody 14 (FIG. 2C).

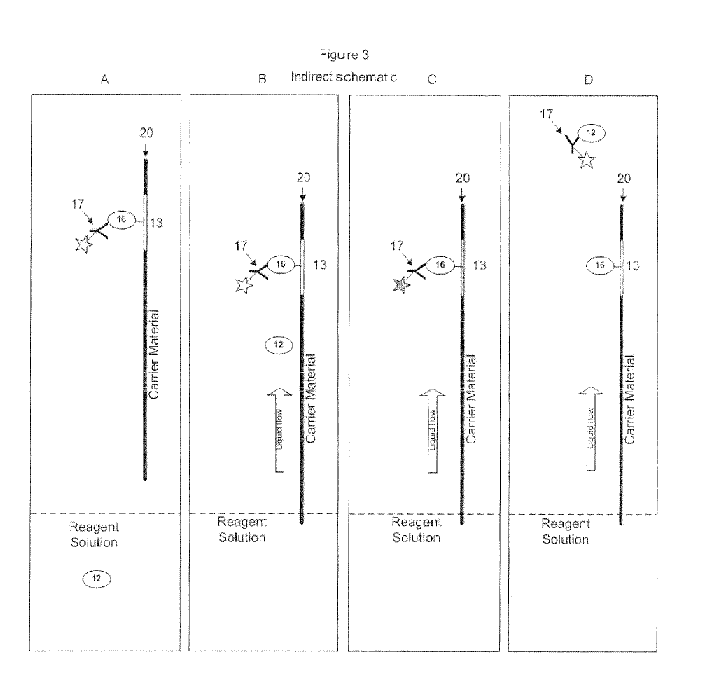

In another embodiment of the invention, referring to FIG. 3, a carrier material 20 comprises an unlabeled triazine moiety 16. The unlabeled triazine moiety 16 is coupled to the carrier material 20 at a detection region 13. In the embodiment, also coupled to the carrier material 20 at region 13 is a labeled antibody 17 bound to the unlabeled triazine moiety 16. Antibody 17 specifically binds to the triazine moiety. Upon addition of a sample, the sample and the reagent solution will migrate through the carrier material 20 to the detection region 13, for example, by capillary action. Triazine moiety 12 present in a sample and which is specifically bound by labeled antibody 17 will compete with the triazine moiety 16 on the carrier material for binding to labeled antibody 17 (FIG. 3D). Such a competition will result in a decrease in the intensity of the signal observed in comparison with the signal that is observed in the absence of triazine moieties in the sample. In the absence of a triazine moiety 12 in a sample, there will not be a competition for binding to labeled antibody 17 (FIG. 3C).

In an embodiment of the invention, the detection device is self-contained in a single casing that encloses all components of the device. The casing can be plastic. However, other suitable inert materials may be used. In an embodiment, the casing is impervious to moisture and inert. In a further embodiment, the casing, in itself, is hollow. It is understood that the single casing may be formed by multiple and/or modular pieces that are assembled together to form the single casing.

In a further embodiment, the detection device is self-contained with non-hazardous substances and thus can be operated without the use of personal safety equipment. Furthermore, in an embodiment, the device is designed to be stable at from 5 to 45° C. and in another embodiment, at around room temperature. The invention involves minimal steps to be performed by the user and is suitable for a use outside of the laboratory settings or environment. The detection device obviates the need for any specialized instruments such as delivery devices like pipettes or visible signal reading devices such as a photometer. Moreover, the detection device is capable of accommodating a consumer product present in a plurality of matrices such as solid, liquid and semi-solid. The detection device is further designed to decrease interference caused by particulate substances that may be present in the sample.

In a further embodiment of the invention, the devices and methods have the advantage of providing a rapid quantitative positive/negative result. This is in contrast to more expensive to manufacture, difficult to use and time consuming quantitative ELISA assays and mass spectrometry devices.

In a further embodiment, the devices and methods provide detection that is highly sensitive and therefore decreases the amount of false negative results. In a further embodiment, the devices and methods permit an indication of a positive result only when the amount of the triazine moiety is above a certain threshold concentration in the test sample. This aspect of the invention is advantageous, because it is useful to have a negative indication if a sample contains only trace elements of a triazine moiety. For example, melamine is a common ingredient in plastics and other materials that frequently come into contact with food and, therefore, trace amounts of melamine are likely found in a number of foods but in amounts that are not harmful to humans or animals. Similarly melamine is found in the environment due to its common industrial use, and therefore it is likely that small amounts of industrial melamine will have leached into food products, but in an amount that is too low to be harmful to humans or animals. Accordingly, it is an aspect of the invention to provide sensitive positive/negative indication of the presence of triazine moieties in a sample above a predetermined threshold concentration, such as below 1 part per million for which the FDA has reported as being an acceptable melamine contaminant level.

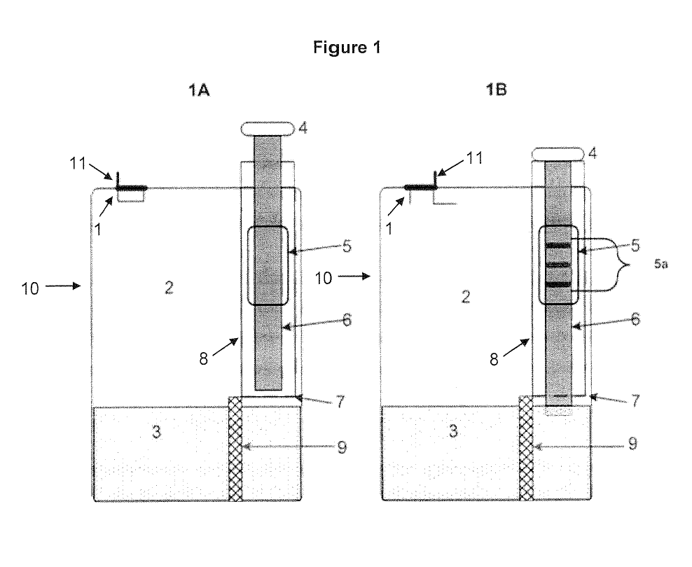

Referring to FIG. 1, in an embodiment of the invention, is a detection device before use (FIG. 1A) and upon activation by the user (FIG. 1B). In this embodiment, the device comprises a casing 10 having a sample receiving port 1 capable of accommodating a pre-determined amount of a sample. Design of the sample receiving port obviates the need for accurately weighing or transferring a solid, semi-solid or liquid sample. The sample receiving port further comprises a cover 11, which receives sample in an open position as shown in FIG. 1A. Upon addition of a sample, the cover 11 is moved to a closed position as shown in FIG. 1B which introduces the sample into a sample mixing area 2.

Referring to the sample receiving port, as discussed above, the port permits detection of an analyte in a liquid, solid or semi-solid state. This is a significant advancement over conventional immunoanalytical detection methods which require that sample be carefully weighed or pipette and pretreated to render it in liquid phase with the amount of solid dissolved in the sample at a predetermined concentration. In contrast, the port of the present invention provides for the introduction of solid or semisolid sample without pretreatment, measuring or dissolution.

The sample mixing area 2 is enclosed within the casing 10. A sample added to the sample receiving port 1 is mixed by, for example, inversion with a reagent solution 3 in the sample mixing area 2. A carrier material 6 is enclosed within a casing 8 and the casing 8 is further enclosed in the casing 10. The carrier material 6 enclosed in the casing 8 is separated from the reagent solution, for example, by a liquid-proof break-away seal 7 before the device is operated by a user. In an embodiment, the sample mixing area 2 is physically separated from the carrier material 6 by inert material such as plastic. It is noted that this compartmentalization of the carrier material from the reagent solution assists the device in detecting multiple matrix forms.

In a further embodiment, filler material may be used, for example, between the sample mixing area and the casing containing the carrier material, in order to reduce volume to facilitate ease of mixing of sample and reaction solution.

Further, a semi-permeable layer such as a membrane, a mesh, a fine mesh or a screen 9 is positioned within the casing 10 to filter the reagent solution containing sample, for example, to limit insoluble particulate matter of a predetermined size from coming into contact with the carrier material 6, but at the same time permitting soluble or dissolved portions of the sample smaller than the predetermined size to come in contact and interact with the carrier material 6. In an embodiment, the semi-permeable layer is designed to permit passage of a predetermined particulate size of melamine cyanurate in crystal form.

In an embodiment, a sample, present in any solid, liquid or semi-solid form, is added to the sample receiving port 1 followed by closure of the sample receiving port using a cover 11. After allowing the sample to be mixed with the reagent solution in the sample mixing area 2, a user will activate a movable piece 4 to pierce the liquid-proof seal 7 permitting contact between the carrier material 6 and the sample mixed with the reagent solution (FIG. 1B).

In a further embodiment, the sample and the reagent solution transport to the carrier material 6 and in a flow path defined by the carrier material to the detection region 5a by capillary action, absorption, wicking, wetting or other flow forces and produce a visible result in detection region 5a.

In a further embodiment, the device contains a test site(s) and a control site(s) in the detection region that are visible to the user through a window 5. In a further embodiment one or more test sites, for example, one or more bands (FIG. 1) or 13 (FIG. 2 and FIG. 3), provide an indication of the presence or absence of an analyte moiety at a predetermined amount. In a further embodiment, the device contains one or more control sites, for example, one or more control bands. In a further embodiment, the control sites include a reference control site(s) and a reagent control site(s). In an embodiment, one or more reference control sites produce a visible color which aids the user in comparing the color intensity produced by the test site. In a further embodiment, one or more control sites confirm that the reagents contained in the reagent solution and/or on the carrier material are functioning properly.

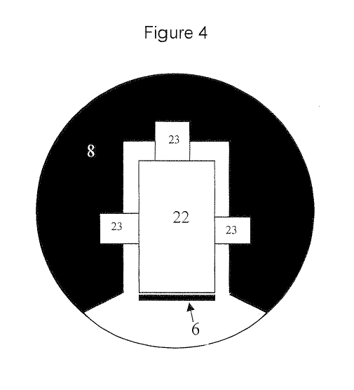

Referring to FIG. 4, which is a cross-section, viewed from above, of an embodiment of a casing and solid support for the carrier material. In this embodiment, enclosed within a casing 8 is a movable solid support 22 having attached or fused to it wings 23. Onto this solid support is attached a carrier material 6. The carrier material is positioned such that a test site (and control site(s)) are viewable to the user. In an embodiment, wings 23 fit into the grooves in the casing 8. In an embodiment, wings 23 and the grooves allow movement of the solid support 22 vertically but with limited (undesired movement) in other directions. In addition to providing fluid movement, this aspect of the invention also provides for less wear and tear on the device. This embodiment further provides for an increased flow direction of diluted sample (reagent solution mixed with the sample) through the carrier material and a decreased flow direction over the other areas of the solid support 22.

In an embodiment, the solid support (and carrier material) has a width of less than 0.5 to 2.5 cm and a height of less than 5 to 20 cm or 10 cm and the solid support has a depth of less than 2.5 cm.

In another embodiment, when the detection device is activated by a user, the wings attached to the solid support are designed to break a waterproof seal that separates the reagent solution and the carrier material allowing flow of the reagent solution mixed with the sample through the carrier material. In an embodiment, the solid support, the wings and the casing are made up of an inert material such as plastic.

In a further embodiment, the sample mixing area and the carrier material are separated by a partition which is pierced or opened by the user via a movable piece or arrangement (other than piece 4) allowing the reagent solution mixed with sample to be in fluid contact with the carrier material, but providing for the carrier material to remain stationary or to be fixed in place. To elaborate, in a further embodiment, piece 4 is fixed and an alternative piercing or introducing component is added to the device to introduce the dissolved analyte from the sample mixing chamber to the carrier material.

As used herein, the terms “including” or “such as” mean, including without limitation. As used herein, all specific descriptions are intended to include approximations. For example, a description “1 year” also should be read to include “about 1 year” or a description “1 ppm” should be read to include “about 1 ppm.” As used herein, the use of “or” in intended to mean “and/or.” The embodiments described herein are illustrative only and are not intended to be read to limit the inventions herein.

1. A test device for determining the presence of one or more compounds in a sample, the device comprising:
a sample receiving port for receiving a pre-determined amount of the sample,
a sample mixing area
a reagent solution contained in the sample mixing area,
one or more carrier materials spaced apart from the sample mixing area,
wherein the one or more carrier materials comprise one or more compounds and/or one or more antibodies that specifically binds to the one or more compounds,
wherein the reagent solution and the one or more carrier materials are designed such that, when the one or more carrier materials are contacted with the reagent solution and a sample comprising a predetermined amount of the one or more compounds, then a visible signal or visible physical change is produced which, without the use of a photometer, indicates the presence or absence of the one or more compounds at a predetermined concentration in the sample,
wherein the device can determine the presence of the one or more compounds in a solid, liquid and semi-liquid sample and
wherein the device weighs less than 2 kg.

2. The device of claim 1, wherein the device is designed to detect two or more compounds at approximately the same time.

3. The device of claim 1, wherein the device is designed to detect 4 or more compounds at approximately the same time.

4. The device of claim 1, wherein the one or more compounds are selected from the group consisting of adulterants, toxins, allergens, pathogens, pesticides, pharmaceuticals, biopolymers and biotechnology products.

5. The device of claim 1, wherein the sample is selected from the group consisting of food, food ingredients, pharmaceuticals, pharmaceutical intermediates, or drinking water.

6. The device of claim 1, wherein the one or more carrier materials comprise the one or more compounds and the one or more antibodies.

7. The device of claim 1, wherein the visible signal is an increase or decrease in color or a color change.

8. The device of claim 1, wherein at least one of the one or more compounds to be detected is selected from the group consisting of melamine, cyanuric acid, melamine cyanurate, ammeline and ammelide.

9. The device of claim 1, wherein the antibody is monoclonal or polyclonal.

10. The device of claim 1, wherein the antibody is an immunoglobulin or a fragment thereof.

11. The device of claim 1, wherein the predetermined amount of at least one of the one or more compounds in the sample is at least about 1 part per million.

12. The device of claim 1, wherein the device weighs less than 1 kg.

13. The device of claim 1, wherein the device weighs less than 0.5 kg.

14. The device of claim 1, wherein the device is pocket-sized.

15. The device of claim 1, further comprising:
a detection region in which a portion of the carrier material is visible,
a breakable liquid proof seal separating the carrier material from the reagent solution,
a movable piece that can be moved to pierce the liquid proof seal in order to initiate a contact between the carrier material and the reagent solution mixed with the sample,
a semi-permeable layer located between the liquid proof seal and the sample receiving port designed to inhibit insoluble particulate matter of a predetermined size that is contained in the sample from coming into contact with the carrier material, and
wherein the device is contained in the casing as a single self-contained unit.

16. A method for detecting one or more compounds, comprising: detecting the presence or absence of a predetermined amount of one or more compounds in a sample using the device of claim 1, wherein the method is carried out by a person untrained in carrying out an immunoassay and is performed outside of a laboratory.
